SEQUENCE LISTING (3 sequences)

SEQ ID NO: 1 - DNA, 477 bp, Glycine max
  source 1..477: mol_type = genomic DNA
atgggtattttcacattcgaggacgaaatcacctcccctgtggctcctgctactctttacaaagctctagttacagatgccgacaacatcatcccaaaggctcttgattccttcaagagtgttgaaaacgttgagggaaatggtggccctggaacaatcaagaagataactttcgttgaggatggagaaaccaagtttgtactgcacaagatagaagcagttgatgaggcaaacttgggatatagctacagcgtagtgggtggtgctgcgttgccagacacagcggagaagatcacattccactccaaattggctgctggtcccaatggaggctctgctggcaagctcactgtcgaataccaaaccaaaggagatgctcagcccaaccaagaccaactcaaaactggaaaagccaaggctgatgctctcttcaaggccattgaggcttaccttttggccaatcccgattacaactaa

SEQ ID NO: 2 - DNA, 1050 bp, Glycine max
  source 1..1050: mol_type = genomic DNA
attcacactatggggcatgcaaagtgacattaacggtactacggtagtgcttgcttataagttataaatagagggcactcctctgctaaagagcatctgaattattcacaaactagaagtattattattcttccatttccattaaaaagaaaatatcataaatgggtattttcacattcgaggacgaaatcacctcccctgtggctcctgctactctttacaaagctctagttacagatgccgacaacatcatcccaaaggctcttgattccttcaagagtgttgaaaacgttgagggaaatggtggccctggaacaatcaagaagataactttcgttgagggtactaactcactcactaatcactagattcatttcattattattccatctctacctatctatatgttgcaatgtatgaatggacagatggagaaaccaagtttgtactgcacaagatagaagcagttgatgaggcaaacttgggatatagctacagcgtagtgggtggtgctgcgttgccagacacagcggagaagatcacattccactccaaattggctgctggtcccaatggaggctctgctggcaagctcactgtcgaataccaaaccaaaggagatgctcagcccaaccaagaccaactcaaaactggaaaagccaaggctgatgctctcttcaaggccattgaggcttaccttttggccaatcccgattacaactaatccaattcgatcaacttcatgattatcatcactatctctcttgctttttctgagacttcatatatctgtatctcacttcatcaataaatcaacaaattataataaaggaaattagccatcattagaccttttctttctcttgtgttcattgaatgaaagataatagtccatactcacgtaaaccaaaatttgattgatcatttttggtaaaatttcttctgataacttaattttttttcaatgagacttgaaccagtagaaattttgtttaagacatttttaacttagtgttacttgaatcaacaacatactaatcttaaccagtt

SEQ ID NO: 3 - protein, 158 aa, Glycine max
MGIFTFEDEITSPVAPATLYKALVTDADNIIPKALDSFKSVENVEGNGGPGTIKKITFVEDGETKFVLHKIEAVDEANLGYSYSVVGGAALPDTAEKITFHSKLAAGPNGGSAGKLTVEYQTKGDAQPNQDQLKTGKAKADALFKAIEAYLLANPDYN

(19) United States
(12) Patent Application Publication
(10) Pub. No.: US 2026/0327645 A1
(43) Pub. Date: Oct. 22, 2026

(54) HIGH THROUGHPUT SOYBEAN ROOT DISEASE SCREEN

(71) Applicants: PIONEER HI-BRED INTERNATIONAL, INC., JOHNSTON, IA (US); EMPRESA BRASILEIRA DE PESQUISA AGROPECUARIA, BRASILIA (BR)

(72) Inventors: FABRICIO BARBOSA MONTEIRO ARRAES, BRASILIA (BR); ANA CRISTINA MIRANDA BRASILEIRO, BRASILIA (BR); NORBERT BRUGIERE, PIBRAC (FR); ADRIANA O. FERREIRA, JOHNSTON, IA (US); PATRICIA MESSENBERG GUIMARÃES, BRASILIA (BR); MARIA FATIMA GROSSI DE SA, BRASILIA (BR); ISABELA TRISTAN LOURENÇO-TESSUTTI, BRASILIA (BR); SANDRA MILACH, JOHNSTON, IA (US); CAROLINA VIANNA MORGANTE, BRASILIA (BR); BRUNA MEDEIROS PEREIRA, BRASILIA (BR); JOHN BRYAN WOODWARD, ANKENY, IA (US)

(73) Assignees: PIONEER HI-BRED INTERNATIONAL, INC., JOHNSTON, IA (US); EMPRESA BRASILEIRA DE PESQUISA AGROPECUARIA, BRASILIA, DF (BR)

(21) Appl. No.: 19/114,903

(22) PCT Filed: Sep. 25, 2023
(86) PCT No.: PCT/US2023/075017

§ 371 Date: Mar. 25, 2025

Related U.S. Application Data
(60) Provisional application No. 63/377,072, filed on Sep. 26, 2022.

(51) Int. Cl. C12N 15/82; C12Q 1/6895

(52) U.S. Cl. CPC C12N 15/8285; C12Q 1/6895; C12Q 2600/13

(57) ABSTRACT

Provided are methods for identifying polynucleotides that increase a plants resistance to a pathogen of interest (e.g., root knot nematode) using hairy-roots generated from leaves comprising providing a population of soybean leaves isolated from a soybean plant population comprising plants susceptible to a pathogen of interest, introducing into the population of soybean leaves a candidate resistance polynucleotide, stimulating hairy-root growth from the population of soybean leaves comprising the introduced candidate resistance polynucleotide, inoculating the hairy-roots with at least one pathogen of interest, and measuring the infection level of the at least one pathogen of interest on the inoculated hairy-roots. Additionally, provided are methods for screening plants for resistance to a pathogen of interest using hairy-roots generated from leaves.

### REFERENCE TO SEQUENCE LISTING SUBMITTED ELECTRONICALLY

The official copy of the sequence listing is submitted electronically via Patent Center as an XML formatted sequence listing with a file named 108032_SequenceListing created on Aug. 24, 2023, and having a size of 5283 bytes and is filed concurrently with the specification. The sequence listing comprised in this XML formatted document is part of the specification and is herein incorporated by reference in its entirety.

### FIELD

This disclosure relates to the fields of plant pathology, plant breeding, molecular biology and biochemistry and, specifically, to methods for identifying germplasm and genes to improve disease resistance.

### BACKGROUND

The root-knot nematodes of the genus Meloidogyne are the most widespread pests, attacking almost every type of crop, including monocotyledonous and dicotyledonous herbaceous and woody plants. These nematodes can cause high yield losses and can strongly affect the quality of production. Approximately 2,000 types of plants are susceptible to infection by root-knot nematodes, causing up to 12% of global crop loss annually and reaching as high as 20% for certain crops. Nematode control is difficult because treatment options are often limited to chemical nematicides. Current methods to identify such resistance genes are labor intensive and time-consuming.

Therefore, there is a need to develop methods to identify genes that improve disease resistance in plants. This disclosure provides such methods.

### SUMMARY

Provided herein are methods for identifying polynucleotides that improve or confer resistance to a pathogen of interest (e.g., root knot nematode) comprising providing a population of soybean leaves isolated from a soybean plant population comprising plants susceptible to a pathogen of interest, introducing into the population of soybean leaves a candidate resistance polynucleotide, stimulating hairy-root growth from the population of soybean leaves comprising the introduced candidate resistance polynucleotide, inoculating the hairy-roots with at least one pathogen of interest, and measuring the infection level of the at least one pathogen of interest on the inoculated hairy-roots.

Also provided is a method for identifying candidate genes that improve resistance to a pathogen of interest in soybean comprising crossing a first soybean plant susceptible to at least one pathogen of interest with a second soybean plant resistant to the at least one pathogen of interest to generate a population of soybean plants, isolating the leaves from the soybean plants of the population of soybean plants, stimulating hairy-root growth from the isolated leaves, inoculating the hairy-roots with the at least one pathogen of interest, measuring the infection level of the at least one pathogen of interest on the inoculated hairy-roots, isolating a pathogen susceptible population and a pathogen resistant population, genotyping the hairy-roots or leaves of the pathogen susceptible population and the hairy-roots or leaves of the pathogen resistant population, and identifying genes or genomic regions present or different in the pathogen resistant population and absent in the pathogen susceptible population as candidate resistance genes.

Further provided is a method for identifying polynucleotides that improve resistance to at least one pathogen of interest in soybean comprising providing (i) a first population of soybean leaves isolated from one or more soybean plants susceptible to the at least one pathogen of interest and (ii) a second population of soybean leaves isolated from one or more soybean plants susceptible to the at least one pathogen of interest, introducing a candidate resistance polynucleotide into the first population of soybean leaves, culturing the first and second population of soybean leaves in a growth media to stimulate hairy-root growth, inoculating the hairy-roots with the at least one pathogen of interest, measuring the infection level of the at least one pathogen of interest on the inoculated hairy-roots, and comparing the level of pathogen infection of the hairy-roots of the first population of leaves and the hairy-roots of the second population of leaves and identifying the candidate polynucleotide as a polynucleotide that improves resistance to the at least one pathogen of interest when the level of pathogen infection in the first population is decreased as compared to the level of infection in the second population.

Also provided is a method of identifying a soybean plant or soybean germplasm having resistance or improved resistance to a root disease comprising isolating leaves from plants of a soybean plant population, stimulating hairy-root growth from the isolated leaves, inoculating the hairy-roots with at least one pathogen of interest, measuring the infection level of the at least one pathogen of interest on the inoculated hairy-roots and identifying plants of the soybean plant population having resistance or improved resistance to the at least one pathogen of interest as compared to a control plant.

Further provided is a method of producing a soybean plant or soybean germplasm having resistance or improved resistance to a root disease comprising crossing a first parent soybean plant susceptible to at least one root disease causing pathogen of interest with a second parent soybean plant resistant to the at least one root disease causing pathogen of interest to generate a population of soybean plants, isolating the leaves from soybean plants of the population of soybean plants, stimulating hairy-root growth from the isolated leaves, inoculating the hairy-roots with the at least one root disease causing pathogen of interest, measuring the infection level of the at least one pathogen of interest on the inoculated hairy-roots, and selecting plants having resistance or improved resistance to the at least one root disease causing pathogen of interest as compared to a control plant.

Provided is a method for characterizing the susceptibility of a plant to a root disease comprising isolating leaves from plants of a soybean plant population, stimulating hairy-root growth from the isolated leaves, inoculating the hairy-roots with at least one root disease causing pathogen of interest, and measuring the infection level of the at least one root disease causing pathogen of interest on the inoculated hairy-roots. In certain embodiments, the method further comprises assigning the plant population a disease score based upon the level of infection of the at least one pathogen of interest.

Further provided is a method to validate and/or select specific single-guide RNAs (sgRNAs) used for genome editing. In certain embodiments, sgRNA molecules designed to trigger the knockout of disease susceptibility genes are identified. In certain embodiments, sgRNA molecules designed to modulate the transcription of specific resistance/susceptibility genes naturally expressed in plant roots are identified.

### BRIEF DESCRIPTION OF THE DRAWINGS AND THE SEQUENCE LISTING

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.831-1.835.

FIGS. 1A, 1B, and 1C provide experimental results depicting the induction of hairy-roots in leaves detached from soybean at various growth stages. FIG. 1A depicts the induction from a complete trefoil. FIG. 1B depicts the induction from the central leaflet isolated from the first full expanded trefoil from the top of the plant at development stage V3. FIG. 1C depicts the induction from unileaves isolated at development stage V1/V2.

### DETAILED DESCRIPTION

Provided herein is a high-throughput method for identifying germplasm having resistance to plant pathogens and polynucleotides and genes conferring resistance to plant pathogens, such as, for example soilborne pathogens. The methods involve stimulating hairy-root growth from plant leaves and expressing a candidate resistance gene. The methods also involve determining if the candidate gene increases resistance to the pathogen by inoculating the hairy-roots with the pathogen of interest and measuring the infection level.

Accordingly, one aspect of the disclosure provides a method for identifying genes that increase resistance to a pathogen of interest and/or confer resistance to a pathogen of interest comprising providing a population of plant leaves (e.g., soybean leaves) expressing a candidate polynucleotide of interest, culturing the population of leaves comprising the introduced candidate resistance polynucleotide in a growth media to stimulate hairy-root growth, inoculating the hairy-roots with at least one pathogen of interest, and measuring the infection level of the at least one pathogen of interest on the inoculated hairy-roots.

In certain embodiments, the method comprises (a) providing a population of leaves isolated from a plant population comprising plants susceptible to a pathogen of interest, (b) introducing into the population of soybean leaves a candidate resistance polynucleotide, (c) stimulating hairy-root growth from the population of soybean leaves comprising the introduced candidate resistance polynucleotide (e.g., culturing the population of leaves comprising the introduced candidate resistance gene “in a growth media to stimulate hairy-root growth), (d) inoculating the hairy-roots with at least one pathogen of interest, and (e) measuring the infection level of the at least one pathogen of interest on the inoculated hairy-roots and comparing the infection level to the infection level in a control sample (e.g., leaves from the same genetic background not expressing the candidate resistance polynucleotide).

In certain embodiments, the method comprises (a) providing (i) a first population of leaves isolated from one or more plants (e.g., soybean) susceptible to the one or more pathogens of interest and (ii) a second population of leaves isolated from one or more plants (e.g., soybean) susceptible to the one or more pathogens of interest, (b) transforming the first population of leaves with a candidate resistance polynucleotide and the second population with a control polynucleotide (e.g., empty vector or EGFP), (c) stimulating hairy-root growth from the first population of soybean leaves and the second population of soybean leaves (e.g., culturing the first and second population of leaves in a growth media to stimulate hairy-root growth), (d) inoculating the hairy-roots with one or more pathogens of interest, (e) measuring the infection level of the one or more pathogens of interest on the inoculated hairy-roots, and (f) comparing the level of pathogen infection between the first and second population of leaves and identifying the candidate polynucleotide as a polynucleotide that improves resistance to the pathogen of interest when the level of pathogen infection in the first population is decreased as compared to the level of infection in the second population.

As used herein, the term “resistance” refers to an absence or reduction of one or more disease symptoms in a plant caused by a plant pathogen. Resistance can mean that disease symptoms, such as, for example, number of lesions, defoliation, and associated yield loss, are reduced, minimized or lessened, when compared to a plant that is susceptible to the disease or a plant that does not contain an effective resistance gene. Further, resistance can include the prevention or delay of proliferation of a pathogen (e.g., fungi).

As used herein the term “increase”, “increased” or the like means to improve, enhance, amplify, multiply, elevate and/or raise, thereby reducing one or more disease symptoms. Accordingly, plants (e.g., soybean) exhibit an increased resistance to a pathogen when compared to plants that are susceptible or tolerant to the pathogen.

The candidate resistance polynucleotide for use in the methods described herein is not particularly limited and may be any polynucleotide of interest for which determining the resistance profile is desired. In certain embodiments, the candidate resistance polynucleotide is isolated from plants having natural resistance to the at least one pathogen of interest. In certain embodiments, the candidate resistance polynucleotide is isolated from a plant selected from the group consisting of soybean (e.g., Glycine arenaria, Glycine argyrea, Glycine cyrtoloba, Glycine canescens, Glycine clandestine, Glycine curvata, Glycine falcata, Glycine latifolia, Glycine max Glycine microphylla, Glycine pescadrensis, Glycine stenophita, Glycine syndetica, Glycine soja, Glycine tabacina and Glycine tomentella), cotton (Gossypium barbadense, Gossypium hirsutum), peanut (e.g., Arachis hypogaea), chickpea (e.g., Cicer arietinum), cowpea (e.g., Vigna unguiculata), pepper (e.g., Capsicum annuum), tomato (e.g., Lycopersicon esculentum) and sweet potato (e.g., Ipomoea batatus).

In certain embodiments of the methods described herein, the candidate resistance polynucleotide is a polynucleotide encoding a pathogenesis-related (PR) protein. As used herein a “PR protein” refers to a protein that is induced in plants in response to a pathogen. On the basis of serological and molecular properties, 11 families of closely related PR proteins (PR1-PR11) have been found in most plants studied.

In certain embodiments of the methods described herein, the candidate resistance polynucleotide is a polynucleotide encoding a β-1,3-glucanase, a chitinase, a thaumatin protein, a thaumatin-like protein, a peroxidase, a defensin (e.g., a ribosome-inactivating protein defensin), a thionin, lipid transfer protein, a proteinase inhibitor, an oxalate oxidase, or an oxalate-oxidase like protein.

In certain embodiments of the methods described herein, the candidate resistance polynucleotide is a polynucleotide that functions in RNA silencing and/or post-transcriptional regulation of gene expression (e.g., microRNA (miRNA)).

As used herein the term “polynucleotide” is not intended to limit the present disclosure to polynucleotides comprising DNA or RNA. Such deoxyribonucleotides and ribonucleotides include both naturally occurring molecules and synthetic analogues. The polynucleotides disclosed herein also encompass all forms of sequences including, but not limited to, single-stranded forms, double-stranded forms, hairpins, stem-and-loop structures, and the like. The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The plant for use in the methods described herein is not particularly limited and may be, for example, any plant that produces hairy-roots and is susceptible to the pathogen of interest. Examples of plant species of interest include, but are not limited to, maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), cotton (Gossypium barbadense, Gossypium hirsutum), coconut (Cocos nucifera), olive (Olea europaea), cashew (Anacardium occidentale), macadamia (Macadamia integrifolia), almond (Prunus amygdalus), green beans (Phaseolus vulgaris), lima beans (Phaseolus limensis), and peas (Lathyrus spp.).

In certain embodiments, the plant of the methods described herein is a legume crop species, including, but not limited to, alfalfa (Medicago sativa); clover or trefoil (Trifolium spp.); pea, including (Pisum satinum), pigeon pea (Cajanus cajan), cowpea (Vigna unguiculata) and Lathyrus spp.; bean (Fabaceae or Leguminosae); lentil (Lens culinaris); lupin (Lupinus spp.); mesquite (Prosopis spp.); carob (Ceratonia siliqua), soybean (Glycine max), or tamarind (Tamarindus indica).

In certain embodiments, the method comprises (a) providing a population of soybean leaves isolated from a soybean plant population comprising soybean plants susceptible to a pathogen of interest, (b) introducing into the population of soybean leaves a candidate resistance polynucleotide, (c) stimulating hairy-root growth from the population of soybean leaves comprising the introduced candidate resistance polynucleotide (e.g., culturing the population of soybean leaves comprising the introduced candidate resistance polynucleotide in a growth media to stimulate hairy-root growth), (d) inoculating the hairy-roots with at least one pathogen of interest, and (e) measuring the infection level of the at least one pathogen of interest on the inoculated hairy-roots.

In certain embodiments of the methods described herein, the genetic background of the plants (e.g., soybean) of the plant population are at least 90%, 95%, 96%, 97%, 98%, or 99% identical. In certain embodiments, the plants (e.g., soybean) of the plant population are all from the same genetic background. In certain embodiments, the one or more plants of the first population and the one or more plants of the second population are of the same genetic background. In certain embodiments, the genetic background of the one or more plants of the first population and the one or more plants of the second population are at least 90%, 95%, 96%, 97%, 98%, 99%, or 100% identical.

In certain embodiments of the methods described herein, the population of leaves are isolated from plants (e.g., soybean) that have not reached full maturity. In certain embodiments of the methods described herein, the population of leaves are isolated from plants (e.g., soybean) that are in a vegetative growth stage. In certain embodiments of the methods described herein the leaves are isolated from a population of plants that are at vegetative growth stage 1 or 2 (V1/V2) or vegetative growth stage 3 (V3) or a combination thereof. In certain embodiments, the population of leaves are isolated from plants that are at vegetative growth stage V1/V2.

As used herein, the vegetative growth stages (V stages) are defined by the number of trifoliate leaves that have developed (unrolled) on the main stem, not branches. The vegetative growth stage 1 (V1 stage) occurs with the full opening of the first trifoliate, the V2 stage occurs with the full opening of the second trifoliate, and the V3 stage occurs with the full opening of the third trifoliate.

As used herein, “isolated”, “isolate” and the like is intended to mean having been removed from its natural environment, such as, for example a leaf no longer attached to the stem on a plant. Accordingly, in certain embodiments of the methods described herein, the isolated leaf is removed from the plant by cutting at the petiole-stem junction.

As used herein, “introducing” “introduced” and the like is intended to mean presenting to the leaf the candidate resistance gene in such a manner that the sequence gains access to the interior of a cell of the leaf. The methods of the disclosure do not depend on a particular method for introducing a sequence into the leaf, only that the polynucleotide gains access to the interior of at least one cell of the plant. In certain embodiments of the methods described herein, the introduced candidate resistance gene is expressed in the hairy-roots grown from the leaves introduced with the candidate resistance gene.

In certain embodiments, the candidate polynucleotide of interest is introduced by transformation. The transformation method to introduce the candidate resistance polynucleotide into the leaf is not particularly limited and may be any transformation method known in the art, preferably a transformation method that can simultaneously lead to hairy root formation, such as, for example Agrobacterium rhizogenes-mediated transformation. In certain embodiments, the method comprises using Agrobacterium-mediated transformation to introduce the candidate resistance polynucleotide. For example, in certain embodiments the transformation method uses Agrobacterium rhizogenes. As should be understood by a person of ordinary skill in the art, in embodiments in which the candidate resistance polynucleotide is not introduced into the leaf by Agrobacterium-mediated transformation (e.g., with Agrobacterium rhizogenes) an additional inoculation step, such as, for example inoculation with Agrobacterium rhizogenes, may be necessary to stimulate hairy-root growth. In certain embodiments, the transformation occurs at the petiole of the isolated leaf.

In certain embodiments of the methods described herein, the candidate resistance polynucleotide is introduced as part of an expression construct. As used herein, a “expression construct” “expression vector” “expression cassette” or the like refers to a nucleotide construct designed to allow expression of a gene (e.g., candidate resistance polynucleotide) in a cell and comprises two or more operably linked DNA segments, preferably DNA segments that are not operably linked in nature (i.e., heterologous). Non-limiting examples of DNA segments include a polynucleotide of interest (e.g., candidate resistance polynucleotide) operably linked to regulatory elements, which aid in the expression, autologous replication, and/or genomic insertion of the sequence of interest. Such regulatory elements include, for example, promoters, expression modulating elements (EMEs), termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

In certain embodiments, the expression construct for use in the methods described herein comprises the candidate resistance polynucleotide operably linked to at least one regulatory element. In certain embodiments, the at least one regulatory element comprises a promoter. In certain embodiments, the at least one regulatory element is heterologous to the candidate resistance polynucleotide of interest. In certain embodiments, the at least one regulatory element is a heterologous promoter.

In certain embodiments, the expression construct can include 5′ and 3′ regulatory sequences operably linked to the polynucleotide of the candidate resistance polynucleotide of interest. In certain embodiments, the expression cassette can include in the 5′-3′ direction of transcription, a transcriptional and translational initiation region (e.g., a promoter), a candidate disease resistance polynucleotide, and a transcriptional and translational termination region (e.g., termination region) functional in plants. The regulatory regions (e.g., promoters, transcriptional regulatory regions, and translational termination regions) and/or the candidate disease resistance polynucleotide may be native/analogous to the host cell or to each other. Alternatively, the regulatory regions and/or the candidate disease resistance polynucleotide may be heterologous to the host cell or to each other. In certain embodiments, the expression construct may comprise 2 or more (e.g., 3, 4, 5, 6, 7, 8, 9, or 10 or more) candidate resistance polynucleotides such that each of the candidate polynucleotides are expressed in the hairy-root tissue. Accordingly, in certain embodiments, a pool or group of candidate resistance polynucleotides are tested simultaneously. In certain embodiments, each of the 2 or more candidate resistance polynucleotides are operably linked to the same regulatory element, such as, for example the same heterologous promoter.

As used herein, “operably linked” refers to the association of two or more nucleic acid fragments on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation. For, example, an operable linkage between a candidate resistance polynucleotide and a regulatory sequence (e.g., a promoter) is a functional link that allows for expression of the candidate polynucleotide of interest. Operably linked elements may be contiguous or non-contiguous. When used to refer to the joining of two protein coding regions, operably linked is intended that the coding regions are in the same reading frame.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the candidate disease resistance polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a regulatory region of DNA capable of regulating the transcription of a sequence operably linked thereto. It usually comprises a TATA box capable of directing RNA polymerase II to initiate RNA synthesis at the appropriate transcription initiation site for a particular coding sequence. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026); GOS2 (U.S. Pat. No. 6,504,083), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

The promoter of the recombinant DNA constructs and/or expression cassettes described herein can be any type or class of promoter known in the art, such that any one of a number of promoters can be used to express the various candidate disease resistance polynucleotide sequences disclosed herein, including the native promoter of the polynucleotide sequence of interest. The promoters for use in the recombinant DNA constructs of the invention can be selected based on the desired outcome, such as, for example expression in the generated hairy-roots.

In certain embodiments, the expression cassette comprises a selectable marker gene. Selectable marker genes are utilized for the selection of transformed cells or tissues. The selectable marker gene for the use in the methods and compositions (e.g., expression cassettes) described herein are not particularly limited. Non-limiting examples of selectable marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional examples of suitable selectable marker genes include, but are not limited to, genes encoding resistance to chloramphenicol; methotrexate; streptomycin; spectinomycin; bleomycin; sulfonamide; bromoxynil; glyphosate; and phosphinothricin. Further examples of suitable selectable marker genes include, but are not limited to, reporter genes such as, for example, glutathione-5-transferase (GST), horseradish peroxidase (HRP), chloramphenicol acetyltransferase (CAT), beta-galactosidase, beta-glucuronidase (GUS), luciferase, green fluorescent protein (GFP), HcRed, DsRed, cyan fluorescent protein (CFP), yellow fluorescent protein (YFP), and blue fluorescent protein (BFP).

In certain embodiments, the selectable marker for use in the expression cassettes and/or recombinant DNA constructs described herein is a fluorescent protein such as, for example, GFP, CFP, YFP or BFP. In certain embodiments of the methods described herein, after culturing the population of leaves to stimulate hairy-root growth and before inoculating the hairy-roots with the at least one pathogen of interest the method further comprises detecting the selectable marker in the hairy-roots of the leaves comprising the introduced candidate resistance polynucleotide and/or control sample and isolating the leaves comprising the hairy-roots expressing the selectable marker such that, for example, all hairy-roots introduced with the candidate resistance polynucleotide inoculated expresses the one or more candidate resistance polynucleotides or control expression vector. The method for detecting the selectable marker is not limited and may be any method known in the art for detecting selected markers. For example, when the selectable marker is a fluorescent protein roots expressing the marker can be identified by fluorescent microscopy.

The number of leaves in the population of soybean leaves is not particularly limited and may be any number of leaves that allows for determination of infection level in the hairy-root compared to a control. In certain embodiments, the population of leaves comprises at least 10, 20, 30, 40, 50, 60, 70, 80, 90, 100, 200, 500 or 1000 leaves and no more than 5000, 2500, 1000, 500, 100, 90, 80, 70, 60, 50, or 40 leaves.

As used herein “culturing” refers to maintaining the leaf population in conditions suitable for growth. In certain embodiments of the methods described herein the leaves in which hairy-root formation is induced by, for example, Agrobacterium transformation, are cultured under conditions that stimulate hairy-root growth. Accordingly, in certain embodiments of the methods described herein the step of stimulating hairy-root growth comprises culturing the population of inoculated leaves in a growth media. The culture conditions are not particularly limited so long as the culture conditions allow for hairy-root growth and can be modified or optimized for the particular plant of interest. Similarly, the growth media is not particularly limited so long as the media allows for hairy-root growth from the cultured leaf. Examples of growth media are known in the art and can be optimized for the particular plant of interest to aid in hairy-root growth.

In certain embodiments of the methods described herein, the at least one pathogen of interest comprises a soilborne pathogen. As used herein a “soilborne pathogen” refers to a disease-causing agent which lives in the soil and can infect a plant (e.g., soybean) host. Soilborne pathogens include, for example, fungi, bacteria, and nematodes. In certain embodiments, the one or more pathogens of interest comprises a nematode. In certain embodiments, the nematode is from the genus Meloidogyne (e.g., M. incognita, M. hapla, M. javanica, and M. arenaria), Heterodera (e.g., H. glycines), Pratylenchus, Rotylenchulus, Hoplolaimus, Helicotylenhus, Belonolaimus, Aphelenchoides, Scutellonema, or Tubixaba. In certain embodiments, the one or more pathogens of interest comprises root knot nematode (RKN) or a soybean cyst nematode (SCN).

Methods of measuring, comparing, and determining the level of resistance, immunity and/or susceptibility (e.g., plant disease symptoms) to a disease, such as, for example, RKN, caused by the pathogen are also known in the art. In certain embodiments of the methods described herein, the level of infection is measured by counting the number of galls present on the hairy-root. As used herein, “gall” refers to an abnormal growth that occurs on the hairy-root, such as, for example, a gall that is the result of RKN infection. In certain embodiments, the level of infection is based on the number of galls present in a unit area. For example, in certain embodiments, the level of infection is measured based on the number of galls per gram of hairy root. In certain embodiments of the methods described herein, the level of infection in hairy-roots expressing the candidate resistance polynucleotide is compared to the level of infection in a control hairy root (e.g., non-transformed hairy roots or roots not expressing the candidate resistance polynucleotide). In certain embodiments, a candidate resistance polynucleotide is identified when the level of infection (e.g., number of galls/gram hairy root) in the hairy root transformed with the candidate resistance polynucleotide is decreased by at least 2%, 5%, 10%, 20%, 30%, 40%, 50%, 100% or more as compared with the control hairy root. In certain embodiments of the methods described herein, the level of infection is measured using the scoring system of Passianotto et al. (Mol Breeding, 37:148 (2017)), based on the system proposed by Crochet and Wad (Uniform Soybean Tests Northern States, USDA-ARS (2004)), in which 1=<10% of the root system, with small galls; 2=10-25% of the root system galled, and most being small galls; 3=26-50% of the root system with large galls; 4=51-90% of the root system with large galls; and 5=91-100% of root system with large galls and necrotic roots. The class of resistance/susceptibility to nematode galls was determined based on the average score of gall for each genotype: resistant, score 1.0 to 2.0; moderately resistant, score 2.2 to 3.0; moderately susceptible, score 3.2 to 4.0; susceptible, score 4.2 to 5.0. In certain embodiments of the methods described herein, the level of infection is measured using the nematode reproductive factor which considers the number of eggs and second-stage juveniles. In certain embodiments, a candidate resistance polynucleotide is identified when the nematode reproductive factor in the hairy root transformed with the candidate resistance polynucleotide is decreased by at least 2%, 5%, 10%, 20%, 30%, 40%, 50%, 100% or more as compared with the control hairy root.

Also provided herein are embodiments in which the plant leaves are isolated from plants expressing the candidate resistance polynucleotide. Thus, in certain embodiments, the method comprises providing a population of leaves isolated from plants (e.g., soybean) expressing a candidate resistance polynucleotide, treating the isolated leaves with a compound that stimulates hairy-root growth (e.g., Agrobacterium rhizogenes), culturing the population of leaves in a growth media, inoculating the hairy-roots with at least one pathogen of interest, and measuring the infection level. In certain embodiments, the candidate resistance polynucleotide is introduced into the plant by a transformation method described herein. In certain embodiments, the candidate resistance polynucleotide is native to the plant. The pathogen of interest and candidate resistance polynucleotide may be any pathogen and candidate resistance polynucleotide described herein. In certain embodiments, the pathogen is RKN. The plant may be any plant described herein. In certain embodiments when the candidate resistance polynucleotide is native to the plant, the plant is a plant species derived from the genus Glycine. Examples of Glycine species include, but are not limited to, Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja, Glycine tabacina and Glycine tomentella.

Provided herein is a method for generating hairy-roots from soybean leaves. In certain embodiments the method comprises (a) providing a population of soybean leaves isolated from a population of soybean plants at developmental stage V1/V2 or V3 or a combination thereof and (b) culturing the population of leaves in a growth media to stimulate hairy-root growth. In certain embodiments, the method further comprises after step (a) and before step (b) transforming the population of leaves with an expression construct using a transformation technique that stimulates hairy-root growth (e.g., Agrobacterium rhizogenes-mediated transformation).

Also provided herein are methods for screening plants for resistance to a pathogen of interest. In certain embodiments, the method comprises providing a population of leaves isolated from a population of plants (e.g., soybean), treating the isolated leaves with a compound that stimulates hairy-root growth (e.g., Agrobacterium rhizogenes), culturing the population of leaves in a growth media, inoculating the hairy-roots with at least one pathogen of interest, and measuring the infection level. In certain embodiments, the infection level is compared to the level of infection in leaves from a control plant, such as, for example a plant susceptible to the pathogen (e.g., Williams 82 variety) or a plant having resistance to the pathogen. In certain embodiments, the population of leaves are isolated from a population of plants having a genetic background and further comprising introduced mutations. In certain embodiments, the population of plants is produced by treating a collection of seeds with a mutagen to produce a mutant population of seeds and growing plants comprising one or more introduced mutation from the mutant seed population. In certain embodiments the mutagen is a chemical mutagen such as base analogues, 5-bromo-uracil, 8-ethoxy caffeine, antibiotics, alkylating agents, sulfur mustards, nitrogen mustards, epoxides, ethylenamines, sulfates, sulfonates, sulfones, lactones, azide, hydroxylamine, nitrous acid, and acridines. In certain embodiments, the mutagen is radiation such as x-rays, gamma rays, neutrons, beta radiation, and ultraviolet radiation. In certain embodiments, the mutagen is a gamma ray administered at a dose of at least 50 Gray. The plant may be any plant described herein. In certain embodiments, the plant is a plant species derived from the genus Glycine. Examples of Glycine species include, but are not limited to, Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja, Glycine tabacina and Glycine tomentella. The pathogen of interest may be any pathogen described herein. In certain embodiments, the pathogen is RKN. In certain embodiments, the method further comprises identifying the causative genes or causative mutations leading to the improved resistance to the pathogen of interest.

In certain embodiments the method for identifying candidate resistance genes that improve resistance to one or more pathogens comprises (a) crossing a first soybean plant susceptible to the one or more pathogens with a second soybean plant resistant to the one or more pathogens of interest to generate a population of soybean plants, (b) isolating the leaves from the soybean plants of the population of soybean plants, (c) stimulating hairy-root growth from the soybean plants of the population of soybean plants (e.g., culturing the isolated leaves in a growth media to stimulate hairy-root growth), (d) inoculating the hairy-roots with one or more pathogens of interest, (e) measuring the infection level of the one or more pathogens of interest on the inoculated hairy-roots, (f) isolating a pathogen susceptible population and a pathogen resistant population, (g) genotyping the leaf and/or generated hairy-roots of the pathogen susceptible population and the leaf and/or generated hairy-roots of the pathogen resistant population, and (h) identifying genes or genomic regions in the pathogen resistant population and absent or different in the pathogen resistant population as candidate resistance genes. The pathogen of interest is not particularly limited and may be any pathogen of interest described herein. For example, the pathogen of interest may be RKN. The leaves for use in the method may be isolated from soybean plants at any of the vegetative growth stages described herein. For example, in certain embodiments the leaves are isolated from plants at the V1/V2 stage. The level of infection can be measured using any method known in the art or described herein. For example, determining the number of galls present on the hairy-root.

In certain embodiments, the first soybean plant susceptible to the one or more pathogens is an elite soybean variety. As used herein, an “elite soybean variety” or “elite soybean germplasm” refers to an agronomically superior line that has resulted from many cycles of breeding and selection for superior agronomic performance. Numerous elite lines are available and known to those of skill in the art of soybean breeding. In certain embodiments, the second soybean plant resistant to the one or more pathogens is an exotic soybean variety. As used herein “exotic soybean variety” or an “exotic soybean germplasm” refers to a variety derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

In certain embodiments the method for identifying candidate resistance genes or genomic regions that improve resistance to one or more pathogens comprises DNA sequencing. As used herein, “sequencing” refers to sequencing methods for determining the order of nucleotides in a molecule of DNA. Any DNA sequencing method known in the art can be used in the methods provided herein. Non-limiting examples of DNA sequencing methods useful in the methods provided herein include long-read molecule sequencing, for example, as described in Liu, Y. et al. (2020). Pan-genome of wild and cultivated soybeans. Cell, 182(1), 162-176, Next Generation Sequencing (NGS) technologies, for example, as described in Egan, A. N, et al. (2012) American Journal of Botany 99(2):175-185; genotyping by sequencing (GBS) methods, for example, as described in Elshire, R. J., et al. (2011) PLoS ONE 6(5):e19379; Molecular Inversion Probe (MIP) genotyping, as described, for example, in Hardenbol, P., et al. (2003) Nature Biotechnology 21(6):673-678; or high throughput genotyping by whole-genome resequencing, as described, for example in Huang, X et al., (2009) Genome Research 19:1068-1076.

In certain embodiments, to identify the candidate resistance genes or genomic regions in the pathogen resistant population the method comprises comparing the sequence of the pathogen resistant population to a susceptible genome sequence, identifying positions that are different between the susceptible genome sequence and the consensus sequence, thereby identifying variants in the plant.

In certain embodiments, to identify the candidate resistance genes in the pathogen resistant population the method comprises comparing the gene expression profile of the pathogen resistant population to the gene expression profile of a pathogen susceptible population and identifying genes whose expression in the resistant population is either increased or decreased (e.g., differentially expressed) as compared to the gene expression in the susceptible population, thereby identifying candidate resistance genes. The method to determine the gene expression profile of the respective populations is not particularly limited and may be any expression profiling method know in the art such as, for example, RNA-Seq.

As used herein, “read mapping” refers to the process of aligning short sequence reads to a reference sequence.

As used herein, “consensus sequence” “consensus base sequence” or the like refers to the calculated order of the most frequent residues found at each position of a sequence alignment. In certain embodiments, the consensus base sequence is the most frequent residues found at each position of the sequences generated for the seeds of an individual plant.

In certain embodiments, the identified candidate resistance genes and/or causative mutations are markers genetically linked to quantitative trait loci (QTLs) associated with the resistance to the pathogen of interest. In certain embodiments, the markers are used for identifying and producing plants comprising the QTL associated with plants having increased resistance to the pathogen of interest.

As used herein, “genetically linked” refers to genetic loci that are statistically determined not to assort independently.

Also provided herein are methods for identifying a soybean plant or soybean germplasm having resistance or improved resistance to a root disease. In certain embodiments, the method comprises isolating leaves from plants of a soybean plant population, stimulating hairy-root growth, inoculating the hairy-roots with at least one root disease causing pathogen of interest, measuring the infection level of the at least one root disease causing pathogen of interest on the inoculated hairy-roots, and identifying plants of the soybean population having resistance or improved resistance to the at least one pathogen. The root disease causing pathogen may be any root disease causing pathogen of interest described herein such as, for example, a nematode (e.g., root knot nematode). The population of leaves may be isolated at any growth stage. In certain embodiments, the leaves are isolated at developmental stage V1/V2 or V3. The method for stimulating hairy-root growth may be any method described herein. In certain embodiments, the plants of the plant population comprise an exotic soybean variety, an elite soybean variety, or a combination thereof.

In certain embodiments of the methods for identifying a soybean plant or soybean germplasm having resistance or improved resistance to a root disease the soybean plant population from which the population of soybean leaves are isolated is generated by a method comprising crossing a first parent soybean variety with a second parent soybean variety to produce progeny seed and growing the progeny seed to generate the plant population which comprises progeny plants. In certain embodiments, the first parent soybean variety or second parent soybean variety is an exotic soybean variety. In certain embodiments, the first parent variety or second parent variety is susceptible to the at least one pathogen of interest.

The method for identifying plants having resistance or improved resistance may be any method described herein. In certain embodiments, the plants are compared to a control plant. In certain embodiments, the control plant is tolerant to the at least one root disease causing pathogen of interest and a plant is considered to have resistance or improved resistance to the pathogen when the infection level of the plant is equal to or less than (e.g., decreased by at least 0%, 2%, 5%, 10%, 20%, 30%, 40%, 50%, 100% or more) the infection level of the control plant. In certain embodiments, the control plant is susceptible to the at least one pathogen of interest and a plant is considered as having resistance or improved resistance to the pathogen when the infection level of the plant is less than (e.g., decreased by at least 2%, 5%, 10%, 20%, 30%, 40%, 50%, 100% or more) the infection level of the control plant.

In certain embodiments, the method for identifying a soybean plant or soybean germplasm having resistance or improved resistance to a root disease further comprises selecting the plants having resistance or improved resistance to the at least one root disease causing pathogen of interest. In certain embodiments, selected plant is crossed with a second plant to produce progeny. In certain embodiments, the second plant is a different plant than the selected plant.

Also provided herein is a method for producing a soybean plant or soybean germplasm having resistance or improved resistance to a root disease comprising crossing a first parent soybean plant susceptible to at least one root disease causing pathogen of interest with a second parent soybean plant resistant to the at least one root disease causing pathogen of interest to generate a population of soybean plants, isolating the leaves from the soybean plants of the population of soybean plants, inoculating the hairy-roots with the at least one root disease causing pathogen of interest, measuring the infection level of the at least one pathogen of interest on the inoculated hairy-roots, and selecting plants having resistance or improved resistance to the at least one root disease causing pathogen of interest as compared to a control plant. The root disease causing pathogen may be any root disease causing pathogen of interest described herein such as, for example, a nematode (e.g., root knot nematode). The population of leaves may be isolated at any growth stage. In certain embodiments, the leaves are isolated at developmental stage V1/V2 or V3. The method for stimulating hairy-root growth may be any method described herein.

The method for identifying plants having resistance or improved resistance may be any method described herein. In certain embodiments, the plants are compared to a control plant. In certain embodiments of the method for producing a soybean plant or soybean germplasm having resistance or improved resistance to a root disease, the control plant is the second parent plant and a plant is considered to have resistance or improved resistance to the pathogen when the infection level of the plant is equal to or less than (e.g., decreased by at least 0%, 2%, 5%, 10%, 20%, 30%, 40%, 50%, 100% or more) the infection level of the control plant. In certain embodiments, the control plant is the first parent plant and a plant is considered as having resistance or improved resistance to the pathogen when the infection level of the plant is less than (e.g., decreased by at least 2%, 5%, 10%, 20%, 30%, 40%, 50%, 100% or more) the infection level of the control plant.

In certain embodiments of the method for producing a soybean plant or soybean germplasm having resistance or improved resistance to a root disease, the method further comprises self-pollinating the selected plant having resistance or improved resistance to the at least one root disease causing pathogen to produce a soybean population having resistance or improved resistance. In certain embodiments, the self-pollinated progeny is crossed with a different soybean plant to generate a progeny population comprising plants having resistance or increased resistance to the pathogen.

In certain embodiments of the method for producing a soybean plant or soybean germplasm having resistance or improved resistance to a root disease, the method further comprises crossing the selected plant having resistance or improved resistance with a different plant to generate a progeny population. The plants of the progeny population may be self-fertilized (e.g., selfed) a sufficient number of generations to produce a soybean plant having resistance or improved resistance and a sufficient level of homozygosity and phenotypic stability.

Further provided are methods for characterizing the susceptibility of a plant to a root disease causing pathogen. In certain embodiments, the method comprises isolating a leaf or leaves from a plant or population of plants having the same genetic background, stimulating hairy-root growth from the isolated leaf or leaves, inoculating the hairy-roots with the at least one root disease causing pathogen of interest, and measuring the infection level. In certain embodiments, the method further comprises providing a disease score based on the level of infection of the isolated leaf or leaves. In certain embodiments, the disease score is based on the level of infection of the isolated leaf or leaves as compared to standard checks (e.g., plants whose susceptibility or resistance score to a specific pathogen is known and established). The root disease causing pathogen may be any root disease causing pathogen of interest described herein such as, for example, a nematode (e.g., root knot nematode). The population of leaves may be isolated at any growth stage. In certain embodiments, the leaves are isolated at developmental stage V1/V2 or V3. The method for stimulating hairy-root growth may be any method described herein. The method for measuring infection level may be any method described herein or known in the art to measure the level of infection in roots.

Provided are methods for identifying soybean resistance sources which can be used, for example, in breeding. In certain embodiments, the method comprises isolating leaves from plants of a soybean plant population, stimulating hairy-root growth, inoculating the hairy-roots with at least one root disease causing pathogen of interest, measuring the infection level of the at least one root disease causing pathogen of interest on the inoculated hairy-roots, and selecting plants having resistance to the at least one pathogen. The root disease causing pathogen may be any root disease causing pathogen of interest described herein such as, for example, a nematode (e.g., root knot nematode). The population of leaves may be isolated at any growth stage. In certain embodiments, the leaves are isolated at developmental stage V1/V2 or V3. The method for stimulating hairy-root growth may be any method described herein. The method for determining plants having resistance to the at least one pathogen may be any method described herein or known in the art. In certain embodiments, the soybean plant population comprises plants of the same variety. In certain embodiments, the soybean plant population comprises one or more different varieties. In certain embodiments, the plants of the plant population comprise an exotic soybean variety, an elite soybean variety, or a combination thereof.

In certain embodiments, the method further comprises crossing the selected plant having resistance to the at least one pathogen with an elite soybean variety to produce progeny. In certain embodiments, the elite soybean variety is susceptible to the at least one pathogen. In certain embodiments, hairy-roots are produced from the progeny plants and inoculated with the at least one pathogen to identify resistant progeny plant. In certain embodiments, the resistant progeny plants are self-pollinated a sufficient number of generations to produce a soybean plant having resistance to the at least one pathogen and a sufficient level of homozygosity and phenotypic stability.

Also provided herein is a method for screening for introduced genome modifications comprising providing a population of soybean leaves isolated from a soybean plant population, introducing into the population of soybean leaves a genome editing polynucleotide, wherein the genome editing polynucleotide encodes a polynucleotide (e.g., candidate resistance polynucleotide) or targets and alters expression of a polynucleotide (e.g., candidate resistance polynucleotide), stimulating hairy-root growth from the population of soybean leaves comprising the genome editing polynucleotide; and measuring the expression level of the polynucleotide (e.g., candidate resistance polynucleotide). In certain embodiments, the genome editing polynucleotide introduces an edit that increases expression of the polynucleotide (e.g., candidate resistance polynucleotide). In certain embodiments, the genome editing polynucleotide introduces and edit that decreases expression of the polynucleotide (e.g., candidate resistance polynucleotide). The method for measuring expression of the polynucleotide (e.g., candidate resistance polynucleotide) is not particularly limited and may be any method that can detect expression such as, for example, RT-PCR. In certain embodiments, the polynucleotide is a candidate resistance polynucleotide. The method for introducing the genome editing polynucleotide and stimulating hairy-root growth may be any method described herein or known in the art. The genome editing polynucleotide may be any polynucleotide that expresses or targets the polynucleotide (e.g., candidate resistance polynucleotide), such as, for example sgRNA. In certain embodiments, the method further comprises inoculating the hairy-roots with at least one pathogen of interest; and measuring the infection level of the at least one pathogen of interest on the inoculated hairy-roots. The methods for inoculating the hairy-roots with the at least one pathogen of interest and measuring the infection level may be any method described herein. Similarly, the at least one pathogen of interest may be any pathogen described herein.

In certain embodiments of the methods described herein, the method consists essentially of the recited steps.

The following are examples of specific embodiments of some aspects of the invention. The examples are offered for illustrative purposes only and are not intended to limit the scope of the invention in any way.

### Example 1

This example demonstrates the induction of soybean hairy-root from detached soybean leaves and transient candidate resistance gene transformation via Agrobacterium rhizogenes.

Soybean seeds from the William 82 variety (i.e., RKN susceptible plant) were grown in pots under standard conditions. When the plants were in V1/V2 development stage, which occurs around 15-20 days after germination, the leaves were detached by cutting at the petiole/stem junction.

The detached leaves were transformed with either a candidate polynucleotide of interest (GmPR10) or an empty vector. Briefly, the detached leaves were soaked in a bacterial paste comprising either a K599 wild strain of Agrobacterium rhizogenes transformed with an expression vector (pPZP) containing the candidate resistance polynucleotide GmPR10 (SEQ ID NO: 1) and the EGFP selectable marker gene (GmPR10-EGFP) or a K599 wild strain of Agrobacterium rhizogenes transformed with an expression vector (pPZP) comprising the EGFP selectable marker gene (EGFP). The treated leaves were then cultured in a growth media (natura water) under conditions (27 °C with a 12-hour photoperiod) to allow for hairy-root growth.

Tables 1 and 2 provide a summary of the root growth (Table 1) and transformation efficiency as measured by GFP expression in the hairy-roots from leaves transformed with empty-vector or the candidate resistance polynucleotide.

TABLE 1
Emerging Hairy-Roots

| | Leaves with | Total number | % of transformed |
|---|---|---|---|
| | emerging | of leaves | leaves developing |
| Transformation | hairy-roots | transformed | hairy-roots |
| GmPR10-EGFP | 31 | 36 | 86.1% |
| EGFP | 25 | 36 | 69.4% |

TABLE 2
Transformation Efficiency

| | Leaves with GFP | Total number of | % of |
|---|---|---|---|
| | positive | leaves with | GFP positive |
| Transformation | hairy-roots | hairy-roots | hairy-roots |
| GmPR10-EGFP | 26 | 31 | 83.8% |
| EGFP | 20 | 25 | 80.0% |

Taken together, the results show that 77.8% of the transformed plants developed hairy-roots and of the hairy-roots 82.1% were GFP positive indicating that they were expressing the candidate polynucleotide of interest or the empty vector.

These results demonstrate transformation of leaves to generate hairy-roots expressing a candidate resistance polynucleotide of interest.

### Example 2

This example demonstrates the validation of a candidate root-knot nematode resistance polynucleotide.

Hairy-roots expressing GmPR10 or the empty-vector control were inoculated with approximately 1000-2000 J2-stage individuals of Meloidogyne incognita. After incubation for 30-60 days on a bilayer of germitest paper and vermiculite, the roots were washed and weighed, and the number of galls present on the hairy-roots were counted to determine the number of galls per gram of hairy-root. As shown in Table 3, hairy roots transformed with a GmPR10 containing vector displayed a significant reduction in the number of galls/gram of hairy root tissue as compared to hairy roots transformed with an empty vector.

RT-PCR using total RNA extracted from the transformed hairy roots confirmed that the GmPR10 gene was expressed 77.9-fold higher in roots with the GmPR10 transgene gene over control roots. These results validate GmPR10 as a candidate RKN resistance polynucleotide using the high-throughput disease screen.

TABLE 3
Average Gall Counts per Gram of Hairy-Roots

| | | Mean (galls/gram | | P(T <= t) |
|---|---|---|---|---|
| | N | of root) | STDev | two-tail |
| pPZP-GmPR10 | 19 | 22.8 | 9.9 | 0 |
| pPZP-Empty (control) | 17 | 71.8 | 22.6 | |

A two-tailed t-test assuming unequal variances was performed to evaluate significance.

### Example 3

This example demonstrates the optimization of the leaf stage for hairy-root formation.

Soybean seeds from the William 82 variety (i.e., RKN susceptible plant) were grown in pots under standard conditions. Leaves were detached by cutting at the petiole/stem junction from plants with a complete trefoil, the central foliole of plants at development stage V3, and plants at development stage V1/V2.

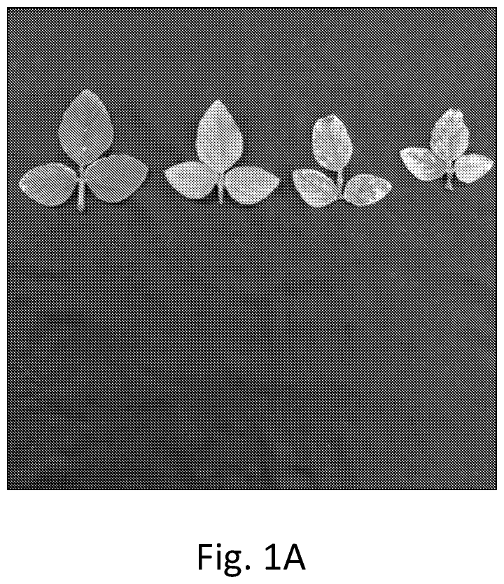

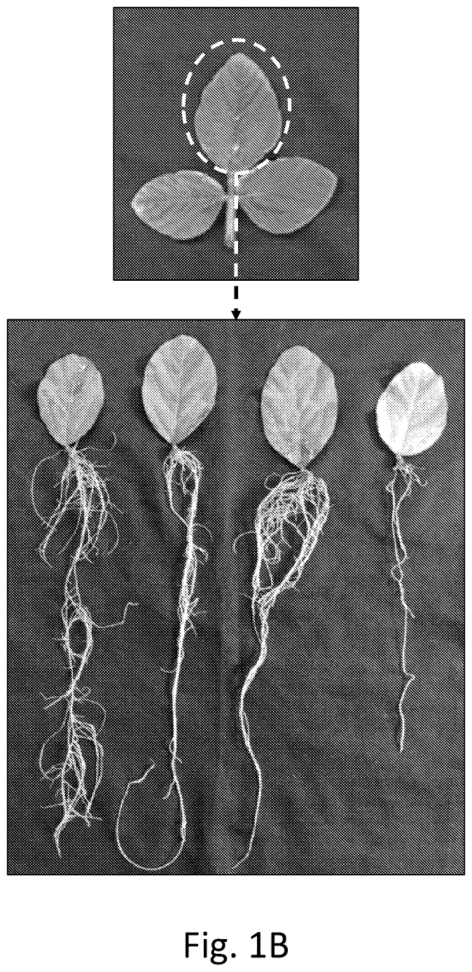

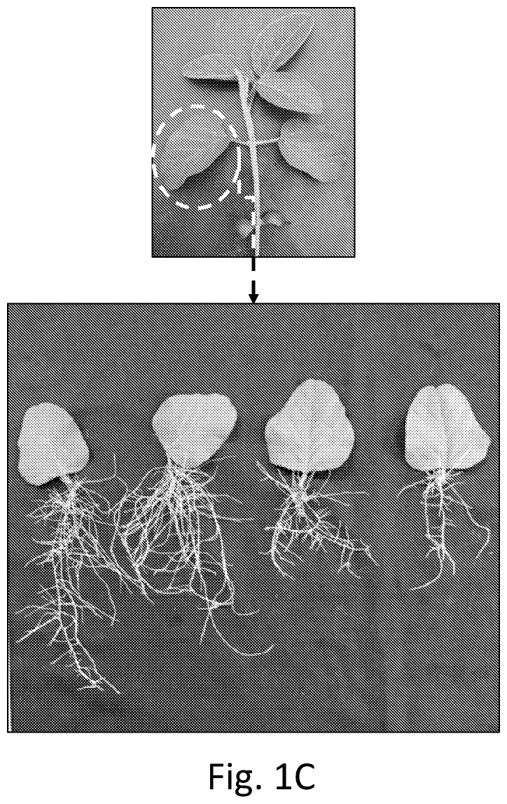

Plants were transformed with an empty vector as described above and hairy-root formation from the transformed plants was determined. As shown in FIGS. 1A-1C, no hairy-roots were formed from the leaves isolated from plants with a complete trefoil (FIG. 1A), whereas hairy-roots were formed from both leaves isolated from plants at development stage V3 (FIG. 1B) and V1/V2 (FIG. 1C). Surprisingly, hairy-roots obtained from the unileaves of the plants at development stage V1/V2 were more voluminous compared to the other stages and were found to be more suitable for bioassays with phytonematodes.

### Example 4

This example demonstrates testing of additional candidate resistance genes. Transcriptomic and proteomic analysis of contrasting soybean genotypes (BRS 133—Susceptible; PI595099—Highly tolerant) infected with M. incognita race 1 revealed differentially expressed genes that could be involved in resistance to root knot nematode (Beneventi, Magda Aparecida, et al. BMC genomics 14.1 (2013): 1-17). Genes differentially expressed between resistant and susceptible genotypes, especially those that are differentially regulated during an infection, are candidate genes for conditioning resistance. Such candidate resistance genes could include, GmGST (Glyma.18G190300; glutathione S transferase), GmFTH1 (Glyma.01G124500; ferritin heavy chain), GmG4DT (Glyma.10G295300; glyceollin), GmREMN (Glyma.09G139200; remorin), and GmUSP (Glyma.04G107900; universal stress protein). Overexpressing these or other candidates in a transient hairy root assay as described above will validate their potential to reduce or prevent root knot-nematode infection. Following Agrobacterium rhizogenes mediated transformation, hairy root establishment, and nematode infection, hairy roots overexpressing the respective candidate genes will be evaluated for the number of galls per gram of hairy root. Candidate genes will be considered as resistance genes when roots overexpressing the gene show a reduced number of galls per gram compared to controls.

### Example 5

This example demonstrates the induction of soybean hairy-root from detached soybean leaves and transcriptional induction of the GmEXPA soybean gene by CRISPR/dCas9 technology.

Soybean seeds from the William 82 variety (i.e., RKN susceptible plant) were grown under standard conditions. When the plants were in V1/V2 development stage, which occurs around 15-20 days after germination, the leaves were detached by cutting at the petiole/stem junction. Detached leaves were transformed with three different sgRNA candidates of interest (sgRNA16, sgRNA26, or sgRNA55) or an empty vector as a control. Each sgRNA recognizes a specific position of the promoter region of the GmEXPA gene (Glyma.06G021700.1), which is known from previous studies to confer resistance to the root-knot nematode M. incognita. The sgRNA16 target the position is −305 from the transcription start site (TSS), the sgRNA26 target position is −184 and the sgRNA55 target position is −157 from TSS. Briefly, detached leaves were inoculated with a bacterial paste containing either a K599 wild strain of Agrobacterium rhizogenes transformed with an expression vector (pMDC32) containing each candidate sgRNA individually, a variation of Cas9 endonuclease (dead Cas9 or dCas9) with deleterious mutations (D10A and H841A) capable of suppressing only the endonuclease activity of the enzyme, the VP64 transcriptional activator, and the EGFP selectable marker gene (sgRN16-EGFP, sgRNA26-EGFP, and sgRNA55-EGFP), or a K599 wild-type strain of Agrobacterium rhizogenes transformed with an expression vector (pMDC32) comprising dCas9, VP64 and the EGFP selectable marker gene (Mock) without any sgRNA polynucleotide. The treated leaves were then cultured in a growth media (natura water) at 27 °C with a 12-hour photoperiod to allow for hairy-root growth.

The expression of the GmEXPA gene, measured by RT-qPCR, was induced by the CRISPR/dCas9 system in soybean hairy-roots, and plants transformed with sgRNA16 and sgRNA55 had higher levels of GmEXPA gene expression, which is linked to the overexpression of the dCas9 (Table 4).

The results summarized in Table 4 showed that hairy-roots induced by the high-throughput disease screen system overexpressing sgRNA16 and sgRNA55 have high levels of the GmEXPA gene and that this expression is linked to the overexpression of the dCas9-VP64 system. This example demonstrates that the detached leaf assay with transformed hairy roots induced by A. rhizogenes could be used to test candidate expression and gene modification systems, such as CRISPR/dCAS9. Additionally, infecting the transformed roots containing modified GmEXPA expression with root knot nematodes and observing reduced galls/gram root tissue, would validate the potential of GmEXPA as a candidate gene for conditioning RKN resistance.

TABLE 4
Relative gene expression of GmEXPA and dCas9 transcripts.

| | | GmEXPA | | dCas9 | |
|---|---|---|---|---|---|
| | | relative | | relative | |
| | N | expression | STDev | expression | STDev |
| sgRNA16-EGFP | 5 | 32.2 | 5.8 | 566.2 | 103.4 |
| sgRNA26-EGFP | 5 | 8.5 | 1.3 | 307.5 | 76.9 |
| sgRNA55-EGFP | 5 | 42.4 | 1.2 | 658.7 | 119.2 |
| Mock | 5 | 1.1 | 0.2 | 0 | 0 |

### Example 6

This example demonstrates the validation of sgRNA designed to induce indels at a specific polynucleotide target using a conventional CRISPR/Cas9 system.

A guide RNA sequence was designed from the CRISPR-P software (Lei, Yang et al. Mol Plant 7.9 (2014): 1494-1496) to induce indels at the soybean Glyma.05G195000 polynucleotide sequence. The selected 20 nucleotides were chemically synthesized and cloned into a modified version of the vector pMDC32, containing the EGFP coding polynucleotide sequence, as a selectable marker gene. Soybean seeds from the William 82 variety were grown in pots under standard conditions. When plants reached V1/V2 development stage, the unileaves were detached at the petiole/stem junction.

The unileaves were inoculated with a bacterial paste comprising a K599 strain of Agrobacterium rhizogenes transformed with a control plasmid vector (pPZP) containing a selectable marker gene (EGFP) or the plasmid of interest in which the sgRNA was cloned. The treated leaves were then cultured in a growth media (natura water) under conditions (27 °C with a 12-hour photoperiod and watering every other day) to allow hairy-root growth. GFP-positive hairy-roots were identified through fluorescence screening.

GFP-positive hairy-roots were collected individually for DNA extraction. The editing target sequence was Sanger sequenced to identify indels. Two out of 24 GFP-positive hairy-roots sequenced were identified with deletions in CRISPR target sequence, representing 8.3% editing efficiency. Here the hairy root method from detached leaves was used to validate a SDN1 system. Infecting the transformed and edited roots with root knot nematodes and observing a difference in galls/gram root tissue would validate Glyma.05G195000 as a candidate gene for conferring resistance to root knot nematodes.

All publications and patent applications in this specification are indicative of the level of ordinary skill in the art to which this invention pertains. All publications and patent applications are herein incorporated by reference to the same extent as if each individual publication or patent application was specifically and individually indicated by reference.

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

1. A method for identifying polynucleotides that improve resistance to a pathogen of interest, the method comprising:
a. providing a population of soybean leaves isolated from a soybean plant population comprising plants susceptible to a pathogen of interest;
b. introducing into the population of soybean leaves a candidate resistance polynucleotide;
c. stimulating hairy-root growth from the population of soybean leaves comprising the introduced candidate resistance polynucleotide;
d. inoculating the hairy-roots with at least one pathogen of interest; and
e. measuring the infection level of the at least one pathogen of interest on the inoculated hairy-roots.

2. The method of claim 1, wherein the soybean leaves are isolated from the plant population by cutting at the petiole-stem junction.

3. The method of claim 1, wherein the candidate resistance polynucleotide is introduced into the petiole of the isolated soybean leaves.

4. The method of claim 1, wherein the candidate resistance polynucleotide is expressed in the hairy-roots.

5. The method of claim 1, wherein the at least one pathogen of interest comprises a soilborne pathogen or a nematode.

6. (canceled)

7. The method of claim 5, wherein the nematode is a root knot nematode (RKN).

8. The method of claim 1, wherein the population of soybean leaves are isolated from the soybean plant population when the soybean plants are at development stage V1/V2 or V3.

9-10. (canceled)

11. The method of claim 1, wherein the candidate resistance polynucleotide is introduced using an expression construct comprising the candidate resistance polynucleotide of interest operably linked to at least one regulatory element.

12. (canceled)

13. The method of claim 11, wherein the at least one regulatory element is heterologous to the candidate resistance polynucleotide of interest.

14. (canceled)

15. The method of claim 11, wherein the expression construct further comprises a selectable marker.

16. The method of claim 15, wherein the selectable marker is a fluorescent protein.

17. The method of claim 15, wherein after step (c) and before step (d) the method further comprises detecting the selectable marker in the hairy root and isolating the leaves comprising hairy roots expressing the selectable marker.

18. The method of claim 1, wherein the candidate resistance polynucleotide is a pathogenesis-related (PR) protein.

19. The method of claim 1, wherein the candidate resistance polynucleotide encodes a β-1,3-glucanase, a chitinase, a thaumatin protein, a thaumatin-like protein, a peroxidase, a defensin, a thionin, lipid transfer protein, a proteinase inhibitor, an oxalate oxidase, or an oxalate-oxidase like protein.

20. The method of claim 1, wherein the level of infection is measured by counting the number of galls present on the hairy-root.

21. (canceled)

22. A method for identifying candidate genes that improve resistance to a pathogen of interest in soybean, the method comprising:
a. crossing a first soybean plant susceptible to at least one pathogen of interest with a second soybean plant resistant to the at least one pathogen of interest to generate a population of soybean plants;
b. isolating the leaves from the soybean plants of the population of soybean plants;
c. stimulating hairy-root growth from the isolated leaves;
d. inoculating the hairy-roots with the at least one pathogen of interest;
e. measuring the infection level of the at least one pathogen of interest on the inoculated hairy-roots;
f. isolating a pathogen susceptible population and a pathogen resistant population;
g. genotyping the hairy-roots or leaves of the pathogen susceptible population and the hairy-roots or leaves of the pathogen resistant population; and
h. identifying genes or genomic regions present or different in the pathogen resistant population and absent in the pathogen susceptible population as candidate resistance genes.

23. The method of claim 22, wherein the at least one pathogen of interest comprises a soilborne pathogen or a nematode.

24. (canceled)

25. The method of claim 23, wherein the nematode is a root knot nematode (RKN).

26. The method of claim 22, wherein the leaves are isolated when the soybean plants of the population of soybean plants are at development stage V1/V2 or V3.

27. (canceled)

28. The method of claim 22, wherein the level of infection is measured by counting the number of galls present on the hairy-root.

29-100. (canceled)
